(19) United States
(12) Patent Application Publication
(10) Pub. No.: US 2021/0289827 A1
(43) Pub. Date: Sep. 23, 2021

(54) FOOD

(71) Applicant: Hanpeng QU, Changsha (CN)

(72) Inventors: Yun LIANG, Changsha (CN); Sheng CAO, Changsha (CN); Shenjian WANG, Changsha (CN)

(21) Appl. No.: 17/343,034

(22) Filed: Jun. 9, 2021

Related U.S. Application Data
(63) Continuation of application No. PCT/CN2019/101025, filed on Aug. 16, 2019.

(30) Foreign Application Priority Data
Dec. 10, 2018 (CN) 201811506528.0

(51) Int. Cl. A23L 33/155; A23L 33/12; A23D 9/007; A23D 9/04; A23D 9/06; A23L 31/00; A23G 3/36; A23G 3/40; A61K 9/00; A61K 31/592; A61K 47/22; A61K 47/24; A61K 47/46; A61K 31/202; A61K 31/07; A61K 36/07; A61K 36/535; A61K 36/52; A61K 36/48; A61K 36/63; A61K 31/734

(52) U.S. Cl. CPC A23L 33/155; A61K 2236/15; A23D 9/007; A23D 9/04; A23D 9/06; A23L 31/00; A23G 3/368; A23G 3/40; A61K 9/0056; A61K 31/592; A61K 47/22; A61K 47/24; A61K 47/46; A61K 31/202; A61K 31/07; A61K 36/07; A61K 36/535; A61K 36/52; A61K 36/48; A61K 36/63; A61K 31/734; A23V 2002/00; A61K 2236/30; A23L 33/12

(57) ABSTRACT

A food contains a vitamin D₂ mushroom oil. A content of vitamin D₂ in unit dose of the food is not less than 0.5 μg. The vitamin D₂ mushroom oil has a peroxide value less than or equal to 15 meq/kg and an acid value less than or equal to 3 mg/g. The vitamin D₂ mushroom oil is prepared through a step of subjecting a vitamin D₂-containing mushroom powder to extraction with an edible oil. The food contains a nutrient selected from the group consisting of perilla oil, walnut oil, linseed oil, peanut oil, olive oil, arachidonic acid (ARA), docosahexaenoic acid (DHA), vitamin A, calcium alginate and a combination thereof.

### CROSS-REFERENCE TO RELATED APPLICATIONS

This application is a continuation of International Patent Application No. PCT/CN2019/101025, filed on Aug. 16, 2019, which claims the benefit of priority from Chinese patent application No. 201811506528.0, filed on Dec. 10, 2018. The content of the aforementioned applications, including any intervening amendments thereto, are incorporated herein by reference.

### TECHNICAL FIELD

This application relates to food production, and more particularly to a food containing a vitamin D₂ mushroom oil.

### BACKGROUND

Vitamin D is an essential vitamin for a human body, and a lack of the vitamin D will cause rickets in children and osteomalacia in adults. The vitamin D can be used for treatments of vitamin D deficiency, chronic hypocalcemia, hypophosphatemia, rickets, osteomalacia with chronic renal insufficiency, familial hypophosphatemia, hypoparathyroidism, acute, chronic and potential postoperative tetany and idiopathic tetany.

The vitamin D is ingested by the human body through food supplements and sunbathing. However, natural foods usually contain a low content of the vitamin D, lean meat, milk, and nuts contain trace amounts of the vitamin D, and vegetables, grains, fruits and their products contain little or no vitamin D. In addition, people with skin diseases and nightlife fail to supplement the vitamin D through sunbathing.

The vitamin D mainly comprises vitamin D₂ (ergocalciferol) and vitamin D₃ (cholecalciferol), where the vitamin D₂ is obtained from plants and the vitamin D₃ is obtained from animals. The existing vitamin D supplements mainly contain the vitamin D₃ from animals. The vitamin D₂ and the vitamin D₃ have similar structures and similar metabolites, whose active metabolites are calcifediol and calcitriol, respectively. Animal pharmacology and toxicology studies show that there is no significant difference between the vitamin D₂ and the vitamin D₃ in stimulating the intestinal absorption of calcium and phosphorus in rats, while a median lethal dose (LD₅₀) of the vitamin D₂ in the rats is about 5-15 times higher than that of the vitamin D₃. Such studies indicate that the vitamin D₂ is safer than the vitamin D₃.

Mushrooms also contain the vitamin D₂, which are processed into mushroom powder for food or medicine in recent years. The mushroom powder has a particle size with 100-200 mesh through drying and crushing by a grinder. However, the mushrooms themselves have lignified fibers and are subjected to the drying process, causing the obtained mushroom powder is solid, insoluble powder. Such mushroom powder can only be applied in solid products and is not easily absorbed by the human body. At the same time, the vitamin D₂ in the mushrooms is fat-soluble vitamin with a low self-dissolution rate, which is not easily absorbed and utilized by the human body through a conventional eating method.

### SUMMARY

An object of this application is to provide a food to solve problems in the prior art that the vitamin D₂ in mushroom powder is not easy to be absorbed and has a low dissolution rate. The food provided herein contains a vitamin D₂ mushroom oil, which is prepared by dissolving the mushroom vitamin D₂ in an edible oil, facilitating promoting the absorption of vitamin D₂ in human body.

Technical solutions of this application are described as follows.

This application provides a food, comprising a vitamin D₂ mushroom oil.

In an embodiment, the vitamin D₂ mushroom oil is used as an ingredient of the food; and a content of vitamin D₂ in unit dose of the food is not less than 0.5 μg, preferably not less than 5 μg, and more preferably not less than 10 μg.

In an embodiment, the vitamin D₂ mushroom oil has a peroxide value less than or equal to 15 meq/kg and an acid value less than or equal to 3 mg/g.

In an embodiment, the vitamin D₂ mushroom oil is prepared through a step of:

subjecting a vitamin D₂-containing mushroom powder to extraction with an edible oil.

In an embodiment, the vitamin D₂ mushroom oil is added with an antioxidant; the antioxidant is selected from the group consisting of: vitamin E, lecithin, rosemary, sodium L-ascorbate and a combination thereof; and an addition amount of the antioxidant is 800-1500 ppm based on a total amount of the vitamin D₂ mushroom oil.

In an embodiment, the edible oil is selected from the group consisting of: sunflower oil, rapeseed oil, soybean oil, olive oil, corn oil, peanut oil, coconut oil, tea seed oil and a combination thereof; a weight ratio of the vitamin D₂-containing mushroom powder to the edible oil is 1:(0.5-30); and the extraction is performed at 10-85° C. for 8-120 h.

In an embodiment, the vitamin D₂-containing mushroom powder is prepared through steps of:

(1) slicing an ergosterol-containing mushroom raw material to obtain mushroom slices; and irradiating the mushroom slices with ultraviolet light for 8-150 min to convert ergosterol into vitamin D₂; and

(2) crushing irradiated mushroom slices to obtain the vitamin D₂-containing mushroom powder with a particle size of 80-150 mesh.

In an embodiment, the food further comprises a nutrient;

wherein the nutrient is selected from the group consisting of perilla oil, walnut oil, linseed oil, peanut oil, olive oil, arachidonic acid (ARA), docosahexaenoic acid (DHA), vitamin A, calcium alginate and a combination thereof.

In an embodiment, the food is a health food or a functional food in a form of a soft capsule, a drop, a spray, a soft candy and a gelatinous candy.

Compared to the prior art, this disclosure has the following beneficial effects.

(1) In this application, the vitamin D₂-containing mushroom powder is replaced with the vitamin D₂ mushroom oil to be added to the food. The vitamin D₂ in the vitamin D₂ mushroom oil is dissolved in the edible oil, improving the dissolution rate of the vitamin D₂ and promoting the absorption of vitamin D₂ in human body.

(2) The vitamin D₂ mushroom oil instead of the vitamin D₂-containing mushroom powder can be applied in the food in forms of the drop and the spray. In addition, the vitamin D₂ mushroom oil does not clog a nozzle, which is more convenient to use the drop and the spray.

(3) The vitamin D₂ mushroom oil of this application is clear and transparent by filtration. Therefore, the vitamin D₂ mushroom oil can be applied in the food in forms of the soft capsule and the gelatinous candy with some degree of transparency, so that their materials inside the gelatin shell are evenly distributed without layering precipitation or eccentricity.

(4) In this application, plant-derived vitamin D₂ is selected to add to the food to supplement vitamin D necessary for the human body instead of animal-derived vitamin D₃, so as to ensure there is no residual solvent or toxic by-product caused by industrial production of the food and a high food safety.

(5) In this application, the vitamin D₂ mushroom oil and the calcium alginate are composited to add to the food, which can provide the human body with vitamin D₂, and also promote the absorption of calcium and growth of bones in children and adolescents and prevent osteoporosis in the elderly.

### DETAILED DESCRIPTION OF EMBODIMENTS

The endpoint values and any values of the range disclosed herein are not limited to the precise range or value, and these ranges or values should be understood to include values close to these ranges or values. For numerical ranges, one or more new numerical ranges may be obtained between the endpoint values of each of the ranges, between the endpoint values of each of the ranges and intermediate values, and between intermediate values. These new numerical ranges shall be considered as specifically disclosed herein.

This application provides a food, containing a vitamin D₂ mushroom oil. An addition amount of the vitamin D₂ mushroom oil is determined according to a content of the vitamin D₂ in the food that meets daily physiological needs of the human body.

In an embodiment, the vitamin D₂ mushroom oil is used as an ingredient of the food. A content of vitamin D₂ in unit dose of the food is not less than 0.5 μg (20 IU), preferably not less than 5 μg (200 IU), and more preferably not less than 10 μg (400 IU). Unit dose of the food represents a measurement value of the smallest package or smallest monomer of the food. For example, the unit dose in a soft capsule is one capsule. The unit dose in a gelatinous candy and a soft candy is one grain. The unit dose in a drop is a single drawing volume of a dropper and the unit dose in a spray is a single spraying volume of a spray nozzle.

In an embodiment, the vitamin D₂ mushroom oil has a peroxide value less than or equal to 15 meq/kg and an acid value less than or equal to 3 mg/g.

In an embodiment, the vitamin D₂ mushroom oil is prepared through a step of:

subjecting a vitamin D₂-containing mushroom powder to extraction with an edible oil.

The vitamin D₂-containing mushroom powder can be obtained by a method provided herein or commercially available. In an embodiment, the vitamin D₂-containing mushroom powder is prepared through the following steps.

(1) An ergosterol-containing mushroom raw material is sliced to obtain mushroom slices. The mushroom slices are irradiated with ultraviolet light for 8-150 min to convert ergosterol into vitamin D₂.

(2) The irradiated mushroom slices are crushed to obtain the vitamin D₂-containing mushroom powder with a particle size of 80-150 mesh. In the step (1), the ultraviolet light is a combination of ultraviolet B (UVB) radiation of 280-315 nm and ultraviolet C (UVC) radiation of 200-280 nm.

The edible oil is edible grease. The edible oil is selected from the group consisting of: sunflower oil, rapeseed oil, soybean oil, olive oil, corn oil, peanut oil, coconut oil, tea seed oil and a combination thereof, where an extraction efficiency of the vitamin D₂ is optimal using the high oleic sunflower oil as an extractant.

An extraction condition only needs to meet that the vitamin D₂ in the vitamin D₂-containing mushroom powder can be extracted and dissolved in the edible oil. In an embodiment, in order to ensure that the vitamin D₂ can be extracted into the edible oil in large quantities, a weight ratio of the vitamin D₂-containing mushroom powder to the edible oil is 1:(0.5-30), preferably 1:(1-10). The extraction temperature is 10-85° C. for 8-120 h, preferably 35-55° C. for 24-72 h. An extraction reaction is preferably carried out under a stirring condition.

The extraction reaction is carried out under an inert gas environment after an extraction tank is vacuumed to protect the vitamin D₂ from being oxidized. The inert gas is preferably nitrogen, more preferably nitrogen with a purity of more than 99.9%. An introduction amount of the inert gas is determined to meet that a tank pressure of the extraction tank is 0.02-0.1 MPa. After the extraction reaction is completed, a solid-liquid separation of the edible oil and the vitamin D₂-containing mushroom powder is performed by filtration, where a filtration method is pressure filtration or suction filtration.

In this application, the vitamin D₂ mushroom oil is added with an antioxidant after the filtration is completed to prevent the vitamin D₂ in the vitamin D₂ mushroom oil from being oxidized. The antioxidant is a commonly used antioxidant in the food. The antioxidant is preferably selected from the group consisting of: vitamin E, lecithin, rosemary, sodium L-ascorbate and a combination thereof, more preferably vitamin E and lecithin. In an embodiment, an addition amount of the antioxidant is 800-1500 ppm based on a total amount of the vitamin D₂ mushroom oil.

The food further contains a nutrient, where the nutrient is selected from the group consisting of perilla oil, walnut oil, linseed oil, peanut oil, olive oil, arachidonic acid (ARA), docosahexaenoic acid (DHA), vitamin A, calcium alginate and a combination thereof to promote mental development of children and adolescents. The vitamin D₂ mushroom oil and the calcium alginate are composited to add to the food, which can promote absorption of calcium by the human body through the vitamin D₂ of the vitamin D₂ mushroom oil.

The food can be various types of food. The food is a health food or a functional food in a form of the soft capsule, the soft candy, the drop, the spray and the gelatinous candy.

A method of preparing the soft capsule containing the vitamin D₂ mushroom oil is performed through the following steps.

(1) Water, gelatin powder and glycerin are mixed and peptized to obtain a sol mixture.

(2) The vitamin D₂ mushroom oil and other components are evenly mixed to obtain a feed liquid and the feed liquid is poured into the sol mixture to obtain the soft capsule.

In an embodiment, an ambient temperature for mixing the vitamin D₂ mushroom oil and other components is preferably lower than 25° C. and a relative humidity is less than 65%. A temperature of the obtained feed liquid is lower than 30° C. In addition, the vitamin D₂ mushroom oil and other components are evenly stirred for 70-80 min. In an embodiment, the obtained feed liquid is preferably subjected to vacuum degassing to avoid excessive contact with oxygen, and the obtained feed liquid should not be stored for more than 2 h under the protection of the inert gas, where the inert gas can be nitrogen. The obtained feed liquid is prepared when it is in need, so as to shorten its exposure time in the air.

In an embodiment, a peptizing temperature is 60-85° C., preferably 70-77° C., a vapor pressure is less than 0.2 MPa and a peptizing time is 40-50 min. A temperature of the sol mixture is controlled at 58-62° C. during discharge and a discharge pressure is less than 0.05 MPa and the sol mixture is filtered through a double-layer bag. In addition, the peroxide value and the acid value of the vitamin D₂ mushroom oil is reduced to reduce the exposure time of the obtained sol mixture in the air, thereby reducing the degree of oxidation and inhibiting the color change of the capsule. The obtained sol mixture should not be stored for more than 48 h, preferably more than 40 h. The obtained sol mixture is more preferably prepared when it is in need.

A method described in step (2) for pouring the feed liquid into the sol mixture can be that for preparing the soft capsule in the prior art, which is not particularly limited in this application.

In a method for preparing the gelatinous candy, a reducing sugar is added to the sol mixture, which is different from the method for preparing the soft capsule.

The soft candy containing the vitamin D₂ mushroom oil is prepared by an existing process, which is not particularly limited in this application.

This application is described in detail with reference to the embodiments below.

In the following preparation methods, the content of the vitamin D₂ in the vitamin D₂ mushroom oil is measured using a high performance liquid chromatograph (L-7000) produced by Hitachi Co., Ltd. according to a GB 5009.82 method. The peroxide value is measured by a GB 5009.227-2016 method, and the acid value is measured by a GB5009.229-2016 method.

The rapeseed oil, the olive oil, the soybean oil and the high oleic sunflower oil are all commercially available products of China Oil And Foodstuffs Corporation. The vitamin E, the rosemary and the sodium L-ascorbate are all commercially available. The perilla oil, the walnut oil, the linseed oil, the peanut oil, the lecithin, the ARA, the DHA, the vitamin A and the calcium alginate are all commercially available, preferably self-made. Agaricus bisporus and Lentinus edodes are commercially available products of Shandong Deze Agricultural Technology Co., Ltd.

The high-purity nitrogen used herein has a purity of more than 99.9%.

### Example 1

(1) 20 kg of freshly-harvested Agaricus bisporus was cleaned to remove the culture medium and cut into slices with a thickness of 1.2 mm. Both sides of the Agaricus bisporus slices were irradiated with UVB with a wavelength of 280 nm at an irradiation dose of 1.5 J/cm² for 120 min and then irradiated with UVC with a wavelength of 200 nm at an irradiation dose of 80 mJ/cm² for 30 min, and then the slices were ground to obtain Agaricus bisporus powder with a particle size of 80-150 mesh.

(2) 20 kg of high oleic acid sunflower oil was fed into an extraction tank under vacuum, to which 2 kg of the Agaricus bisporus powder was sucked. The extraction tank was vacuumized to a micro-positive pressure of 0.06 MPa, and then the high-purity nitrogen was introduced. The Agaricus bisporus powder and the high oleic acid sunflower oil were stirred evenly in the extraction tank and heated to 45° C. The extraction was performed at 45° C. for 72 h, and then extraction mixture was filtered to obtain a filtrate.

(3) The filtrate was added with 14.4 g of vitamin E and 9.6 g of lecithin as antioxidants, evenly stirred and cooled to 25° C. to obtain vitamin D₂ mushroom oil.

### Example 2

(1) 20 kg of freshly-harvested Agaricus bisporus was cleaned to remove the culture medium and cut into slices with a thickness of 1.5 mm. Both sides of the Agaricus bisporus slices were irradiated with UVB with a wavelength of 300 nm at an irradiation dose of 4 J/cm² for 80 min and then irradiated with UVC with a wavelength of 240 nm at an irradiation dose of 100 mJ/cm² for 25 min, and then the slices were ground to obtain Agaricus bisporus powder with a particle size of 80-150 mesh.

(2) 1 kg of rapeseed oil was fed into an extraction tank under vacuum, to which 2 kg of the Agaricus bisporus powder was sucked. The extraction tank was vacuumized to a micro-positive pressure of 0.03 MPa, and then the high-purity nitrogen was introduced. The Agaricus bisporus powder and the rapeseed oil were stirred evenly in the extraction tank and cooled to 10° C. The extraction was performed at 10° C. for 8 h, and then extraction mixture was filtered to obtain a filtrate.

(3) The filtrate was added with 0.48 g of vitamin E and 0.32 g of rosemary as antioxidants, evenly stirred and cooled to 25° C. to obtain vitamin D₂ mushroom oil.

### Example 3

(1) 20 kg of freshly-harvested Lentinus edodes was cleaned to remove the culture medium and cut into slices with a thickness of 0.8 mm. Both sides of the Lentinus edodes slices were irradiated with UVB with a wavelength of 315 nm at an irradiation dose of 6.5 J/cm² for 8 min and then irradiated with UVC with a wavelength of 280 nm at an irradiation dose of 120 mJ/cm² for 20 min, and then the slices were ground to obtain Agaricus bisporus powder with a particle size of 80-150 mesh.

(2) 30 kg of soybean oil and 30 kg of olive oil were fed into an extraction tank under vacuum, to which 2 kg of the Lentinus edodes powder was sucked. The extraction tank was vacuumized to a micro-positive pressure of 0.1 MPa, and then the high-purity nitrogen was introduced. The Lentinus edodes powder and the soybean oil and the olive oil were stirred evenly in the extraction tank and heated to 80° C. The extraction was performed at 80° C. for 120 h, and then extraction mixture was filtered to obtain a filtrate.

(3) The filtrate was added with 90 g of sodium L-ascorbate as antioxidants, evenly stirred and cooled to 25° C. to obtain vitamin D₂ mushroom oil.

Parameters of the vitamin D₂ mushroom oil prepared by the above examples are shown in Table 1.

TABLE 1
Parameters of the vitamin D₂ mushroom oil

| | Content of | Peroxide | Acid |
|---|---|---|---|
| | Vitamin | value | value |
| Sample | D₂ (IU/g) | (meq/kg) | (mg/g) |
| Example 1 | 2363.1 | 3.79 | 0.57 |
| Example 2 | 88465.7 | 4.77 | 0.76 |
| Example 3 | 1606 | 4.32 | 0.72 |

### Example 4 Preparation of Soft Capsule

(1) A gelatin melter and feed and discharge pipelines were sterilized by steam for 15-30 min before production. 11.88 kg of water was boiled and sucked into the gelatin melter. At the same time, 5.6 kg of glycerin, 0.64 kg of microcrystalline cellulose and 11.88 kg of gelatin powder were sucked into the gelatin melter and mixed under stirring. The gelatin melting was performed at a temperature of 75° C. and a vapor pressure of 0.2 MPa for 45 min. After the melting process was completed, the mixture was cooled to 60° C., filtered and subjected to vacuum degassing to obtain a sol for use, where the storage time should not exceed 48 h.

(2) 16.95 kg of the vitamin D₂ mushroom oil prepared in Example 1 was added to a mixing container at an ambient temperature of less than 25° C. and a relative humidity of less than 65%. 4.25 kg of peanut oil, 9.5 kg of linseed oil, 10.5 kg of walnut oil and 8.8 kg of perilla oil were mixed and poured into the mixing container. The mixture in the mixing container was stirred for 75 min and then subjected to vacuum degassing to obtain a feed liquid for use, where the feed liquid should not be stored for more than 2 h.

(3) The feed liquid prepared in step (2) was poured into the sol prepared in step (1) in a pelleter and pelletized at a temperature of 20° C. and a relative humidity of 65% to obtain the soft capsules with a gelatin shell thickness of 0.65-0.8 mm, where a shaping temperature of the gelatin shell was 10° C. An average weight of the gelatin shell of the soft capsules was 300 mg; an average weight of the material inside the gelatin shell was 500 mg; and each soft capsule contained 400 IU of vitamin D₂ and 57 mg of linolenic acid.

The soft capsules were dried at a humidity of 65% and a temperature of 30° C. for 6 h, then dried at a humidity of 30% and a temperature of 32° C. for 6 h and cleaned with food-grade petroleum ether to obtain finished soft capsules. A unit dose of the finished soft capsules was one capsule and the recommended dose was one capsule per day.

### Example 5 Preparation of Soft Capsule

(1) A gelatin melter and feed and discharge pipelines were sterilized by steam for 15-30 min before production. 11.68 kg of water was boiled and sucked into the gelatin melter. At the same time, 6 kg of glycerin, 0.64 kg of microcrystalline cellulose and 11.68 kg of gelatin powder were sucked into the gelatin melter and mixed under stirring. The gelatin melting was performed at a temperature of 75° C. and a vapor pressure of 0.2 MPa for 45 min. After the melting process was completed, the mixture was cooled to 60° C., filtered and subjected to vacuum degassing to obtain a sol for use, where the storage time should not exceed 48 h.

(2) 8.48 kg of the vitamin D₂ mushroom oil prepared in Example 1 was added to a mixing container at an ambient temperature of less than 25° C. and a relative humidity of less than 65%. 15 kg of peanut oil, 3.02 kg of olive oil, 25 mg of vitamin A and 23.5 kg of calcium alginate were mixed and poured into the mixing container. The mixture in the mixing container was stirred for 75 min and then subjected to vacuum degassing to obtain a feed liquid for use, where the feed liquid should not be stored for more than 2 h.

(3) The feed liquid prepared in step (2) was poured into the sol prepared in step (1) in a pelleter and pelletized at a temperature of 20° C. and a relative humidity of 65% to obtain the soft capsules with a gelatin shell thickness of 0.65-0.8 mm, where a shaping temperature of the gelatin shell was 10° C. An average weight of the gelatin shell of the soft capsules was 300 mg; an average weight of the material inside the gelatin shell was 500 mg; and each soft capsule contained 200 IU of vitamin D₂, 100 IU of vitamin A and 80 mg of calcium.

The soft capsules were dried at a humidity of 65% and a temperature of 30° C. for 6 h, then dried at a humidity of 30% and a temperature of 32° C. for 6 h and cleaned with food-grade petroleum ether to obtain finished soft capsules. A unit dose of the finished soft capsules was one capsule and the recommended dose was two capsules per day.

### Example 6 Preparation of Gelatinous Candy

(1) A gelatin melter and feed and discharge pipelines were sterilized by steam for 15-30 min before production. 6.2 kg of water was boiled and sucked into the gelatin melter. At the same time, 2.9 kg of crystalline fructose, 1.2 kg of xylitol, 0.6 kg of microcrystalline cellulose, 4.6 kg of glycerinum and 14.5 kg of gelatin powder were sucked into the gelatin melter and mixed under stirring. The gelatin melting was performed at a temperature of 75° C. and a vapor pressure of 0.2 MPa for 45 min. After the melting process was completed, the mixture was cooled to 60° C., filtered and subjected to vacuum degassing to obtain a sol for use, where the storage time should not exceed 48 h.

(2) 0.226 kg of the vitamin D₂ mushroom oil prepared in Example 2 was added to a mixing container at an ambient temperature of less than 25° C. and a relative humidity of less than 65%. 23.2 kg of DHA, 15.574 kg of peanut oil and 11 kg of walnut oil were mixed and poured into the mixing container. The mixture in the mixing container was stirred for 75 min and then subjected to vacuum degassing to obtain a feed liquid for use, where the feed liquid should not be stored for more than 2 h.

(3) The feed liquid prepared in step (2) was poured into the sol prepared in step (1) in a pelleter and pelletized at a temperature of 20° C. and a relative humidity of 65% to obtain the gelatinous candies with a gelatin shell thickness of 0.65-0.8 mm, where a shaping temperature of the gelatin shell was 10° C. An average weight of the gelatin shell of the gelatinous candies was 300 mg; an average weight of the material inside the gelatin shell was 500 mg; and each gelatinous candy contained 200 IU of vitamin D₂ and 100 IU of DHA.

The gelatinous candies were dried at a humidity of 65% and a temperature of 30° C. for 6 h, then dried at a humidity of 30% and a temperature of 32° C. for 6 h and cleaned with food-grade petroleum ether to obtain finished gelatinous candies. A unit dose of the finished gelatinous candies was one grain and the recommended dose was two grains per day.

### Example 7 Preparation of Spray

27.07 L of the vitamin D₂ mushroom oil prepared in Example 3 was added to a mixing container at an ambient temperature of less than 25° C. and a relative humidity of less than 65%, to which 22.93 L of walnut oil was added. The mixture was mixed for 25 min and poured into food-grade polyester bottles each for 50 mL, where the polyester bottles were provided with a spray nozzle. Single spray dose of the spray nozzle was about 0.5 mL, in which a content of vitamin D₂ was about 400 IU. The unit dose of the spray was a single spraying volume of the spray nozzle, and the recommended dose was 0.5 mL, i.e., the single spraying volume of the spray nozzle.

### Example 8 Preparation of Drop

18.39 L of the vitamin D₂ mushroom oil prepared in Example 1 was added to a mixing container at an ambient temperature of less than 25° C. and a relative humidity of less than 65%, to which 18.7 L of walnut oil and 12.91 L of perilla oil were added. The mixture was mixed for 25 min and poured into food-grade polyester bottles each for 50 mL, where the polyester bottles were provided with a dropper. Single drop dose of the dropper was about 0.5 mL, in which a content of vitamin D₂ was about 400 IU. The unit dose of the drop was the volume drawn by the dropper each time, and the recommended dose was 0.5 mL, i.e., the single drawing volume of the dropper.

### Example 9 Preparation of Drop

9.2 L of the vitamin D₂ mushroom oil prepared in Example 1 was added to a mixing container at an ambient temperature of less than 25° C. and a relative humidity of less than 65%, to which 21.84 L of walnut oil and 18.96 L of ARA were added. The mixture was mixed for 25 min and poured into food-grade polyester bottles each for 50 mL, where the polyester bottles were provided with a dropper. Single drop dose of the dropper was about 0.5 mL, in which a content of vitamin D₂ was about 200 IU and a content of ARA was 75 mg. The unit dose of the drop was the volume drawn by the dropper each time, and the recommended dose was the volume drawn by the dropper twice, i.e., 1 mL.

### Example 10 Preparation of Soft Candy

(1) 2 kg of agar, 2 kg of carrageenan and 5 kg of white granulated sugar were evenly mixed, to which 20 kg of water was added. The mixture was soaked for 20 min and then heated for complete dissolution to obtain a sol.

(2) 5 kg of white granulated sugar, 5 kg of malt syrup and 6.76 kg of water were mixed, heated, boiled and stirred to form a sugar solution. When the sugar solution was not easy to break off after picked up, the sugar solution was added with the sol prepared in step (1), evenly stirred and then boiled at 104-107° C. When a moisture content of the system dropped to 22-24%, the heating was stopped, and the system was cooled to 70° C., added with 4.24 kg of the vitamin D₂ mushroom oil prepared in Example 1 and evenly stirred to obtain syrup.

(3) The syrup prepared in step (2) was poured into a mold while it was hot. The syrup was cooled and solidified to form a molded sugar body, which was dried by hot air drying at 45° C. for 8-10 h to enable the moisture content to drop to 14-18%. The dried sugar body was cooled to room temperature to obtain the soft candy. An average weight of each soft candy was 500 mg and a content of vitamin D₂ in each soft candy was 100 IU. A unit dose of the soft candies was one grain, and the recommended dose was four grains per day.

The above are only preferred embodiments of this application, which are not intended to limit this application. It should be understood that changes, modifications and replacements made by those skilled in the art without departing from the spirit of this application shall fall within the scope of this application.

What is claimed is:

1. A food, comprising a vitamin D₂ mushroom oil.

2. The food of claim 1, wherein the vitamin D₂ mushroom oil is used as an ingredient of the food; and a content of vitamin D₂ in unit dose of the food is not less than 0.5 μg.

3. The food of claim 1, wherein the vitamin D₂ mushroom oil has a peroxide value less than or equal to 15 meq/kg and an acid value less than or equal to 3 mg/g.

4. The food of claim 1, wherein the vitamin D₂ mushroom oil is prepared through a step of:
subjecting a vitamin D₂-containing mushroom powder to extraction with an edible oil.

5. The food of claim 4, wherein the vitamin D₂ mushroom oil is added with an antioxidant; the antioxidant is selected from the group consisting of: vitamin E, lecithin, rosemary, sodium L-ascorbate and a combination thereof; and an addition amount of the antioxidant is 800-1500 ppm based on a total amount of the vitamin D₂ mushroom oil.

6. The food of claim 4, wherein the edible oil is selected from the group consisting of: sunflower oil, rapeseed oil, soybean oil, olive oil, corn oil, peanut oil, coconut oil, tea seed oil and a combination thereof; a weight ratio of the vitamin D₂-containing mushroom powder to the edible oil is 1:(0.5-30); and the extraction is performed at 10-85° C. for 8-120 h.

7. The food of claim 4, wherein the vitamin D₂-containing mushroom powder is prepared through steps of:
(1) slicing an ergosterol-containing mushroom raw material to obtain mushroom slices; and irradiating the mushroom slices with ultraviolet light for 8-150 min to convert ergosterol into vitamin D₂; and
(2) crushing irradiated mushroom slices to obtain the vitamin D₂-containing mushroom powder with a particle size of 80-150 mesh.

8. The food of claim 1, further comprising a nutrient;
wherein the nutrient is selected from the group consisting of perilla oil, walnut oil, linseed oil, peanut oil, olive oil, arachidonic acid (ARA), docosahexaenoic acid (DHA), vitamin A, calcium alginate and a combination thereof.

9. The food of claim 2, further comprising a nutrient;
wherein the nutrient is selected from the group consisting of perilla oil, walnut oil, linseed oil, peanut oil, olive oil, arachidonic acid (ARA), docosahexaenoic acid (DHA), vitamin A, calcium alginate and a combination thereof.

10. The food of claim 1, wherein the food is a health food or a functional food in a form of a soft capsule, a drop, a spray, a soft candy and a gelatinous candy.
